(19) United States
(12) Patent Application Publication
(10) Pub. No.: US 2024/0167865 A1
(43) Pub. Date: May 23, 2024

(54) LEVEL AND/OR DENSITY SENSOR DEVICE FOR LIQUID VESSELS

(71) Applicant: Watgrid, Lda., Ílhavo (PT)

(72) Inventors: Fábio Patrício Domingues Gonçalves, Santiago De Litém (PT); Lúcia Maria Botas Bilro, Gafanha Da Encarnação (PT); Rogério Nunes Nogueira, Aveiro (PT)

(21) Appl. No.: 18/427,962

(22) Filed: Jan. 31, 2024

Related U.S. Application Data
(63) Continuation of application No. 16/207,514, filed on Dec. 3, 2018, now Pat. No. 11,933,654.

(30) Foreign Application Priority Data
Jun. 14, 2016 (PT) 109451

(51) Int. Cl. G01F 23/14; C12M 1/34; G01F 23/16; G01F 23/18; G01N 9/26

(52) U.S. Cl. CPC G01F 23/14; G01F 23/168; G01F 23/18; G01N 9/26; C12M 41/44

(57) ABSTRACT

The present disclosure is related to a level and/or density sensor for vessels suitable for storing liquids, in particular barrels or vats, more in particular barrels or vats for storing or producing wine. The hydrostatic pressure differential sensor for measuring volume and/or density disclosed herein comprises a main body; a tube for diving into the liquid; a hydrostatic pressure differential sensor; an air injector for injecting air into said tube; wherein the tube is coupled to the main body; wherein the hydrostatic pressure differential sensor has two inlets, a first inlet airtight connected to the upper top of said tube, configured so that the tube maintains air present in the interior thereof when dived into the liquid, and a second inlet for communicating with the interior of the vessel.

### TECHNICAL FIELD

The present disclosure is related to a level and density sensor device for vessels suitable for storing liquids, barrels or vats, more in particular barrels or vats for storing or producing wine.

### BACKGROUND

The wine-making process is a set of technical operations allowing the transformation of grapes into wine and comprising several steps among which wine fermentation, ageing and storage steps in barrels/tuns/casks/vats or other vessels suitable for the purpose.

Offline control by sampling means is the most common form of controlling the wine-making process, namely for controlling the density.

The control of the wine level, when stored in barrels, normally implies opening the barrel, removing the stopper, for verifying the existing space between the top of the barrel and the liquid level.

Controlling the level is essential for ullage, which consists of resetting the level for compensating the losses occurred through gaseous exchange, which usually occurs every 2/3 months. It is estimated that each barrel loses 7 to 11 liters of wine, depending on the environmental conditions.

Gaseous exchanges are directly related to the wine oxygen dissolution, with various redox reactions occurring, which will form unstable compounds, leading to changes in color and wine astringency and contributing to the ethanol oxidation to acetaldehyde, being that the acetaldehyde contributes afterwards to the copolymerization of the flavonoids and anthocyanins.

The existence of an excessive air pocket promotes wine oxidation. Therefore, the wine level control is related to the quantity of available oxygen.

In the alcoholic fermentation step (transformation of sugars into alcohol) measuring the liquid density (must) is one of the ways of controlling the activity of yeasts, allowing for accompanying the fermentation progress.

These facts are described so as to illustrate the technical problem solved by the embodiments of this document.

### General Description

The present disclosure is related to a level and/or density sensor device for vessels suitable for storing liquids, barrels or vats, more in particular barrels or vats for fermenting, storing and producing wine.

Offline control by sampling means is the most common way of controlling the wine-making process. However, this way of control, by sampling, translates into a process that:
must be carried out by an operator on the wine storage location;
consumes time and resources for wine producers, since it is a manual process;
induces an action/reaction cycle causing greater variations, which leads to lower wine quality;
is carried out only for a fraction of the production and consequently, the product quality is not uniform.

The importance of knowing at every moment what is the level and density of the liquid contained in a cask/tun/barrel/vat, relates to the following aspects:

| Variable | | Fermentation |
|---|---|---|
| of | | and/or |
| interest | Barrel | storage reservoir |
| Level | Wine - At the ageing stage it allows | It may detect the |
| | accompanying the evaporation, which is | existence of |
| | directly related to the gaseous exchanges | leakage, unusual |
| | with the exterior, allowing for evaluating | liquid evaporation |
| | the need to carry out ullage. | and stock control. |
| | Spirit drinks - At the ageing stage it | |
| | allows accompanying the evaporation, | |
| | which is directly related to the alcohol | |
| | content of the spirit drink. | |
| Density | Wine - It allows accompanying it in the | Wine - It allows |
| | fermentation stage, that is, the sugar | accompanying it |
| | degradation, being that the yeast activity | in the |
| | is followed so as to transform sugar into | fermentation |
| | alcohol (in the fermentation stage density | stage, that is, |
| | may go from 1100 kg/m³ to 990 kg/m³ at | the sugar |
| | a temperature of 20° C.). | degradation. |
| | Spirit drinks - In the ageing stage it allows | |
| | accompanying the reduction of the | |
| | alcohol content. | |

Controlling the level and density of the liquid contained in a cask/tun/barrel/vat is a complex and lengthy process for being carried out manually, considering, for example, that each barrel/vat may contain a different liquid, or be in a fermentation/ageing stage different than the remaining barrels/vats, that is, there may exist a plurality of different needs for the various barrels/vats. Therefore, this problem is solved with the present disclosure through the implementation of the sensor device disclosed herein in each barrel/vat.

Solutions currently existing in the market present the following limitations: don't allow for an automated/in real time monitoring (buoy solution); present a high cost reducing economic viability; are affected by the deposition of sediments/particles (crystallization effects), needing regular maintenance (capacitive/pressure sensors with membranes in direct contact with the liquid); operation ranges outside the necessary requirements (ultrasounds distances>300 mm) and insufficient resolutions (>1 mm ultrasounds).

The advantages of the present disclosure comprise:
increasing the wine quality;
reducing waste, since the sampling need for chemical analyses and additionally losses from meagre monitoring of the quality, possibly leading to the total loss of the product;
reducing control costs;
possibility of controlling a set of barrels through a network of sensor devices incorporated in a monitoring platform, which translates into control and monitoring in real time;
the physical and chemical integrity of the liquid is not endangered (there is no contamination);
facilitating the maintenance since the particle deposition does not compromise the functioning of the sensor device.

In an embodiment, it is possible to control the following parameters in real time: level, density, temperature, pH, turbidity, color, sugar and alcohol quantity contained in the vessel/barrel/tun/cask/vat, namely using additional sensors.

Embodiments of the present disclosure comprise the possibility of controlling the various parameters more than once; sending notifications when the monitored parameters are outside the limits defined by the user; real time monitoring; wireless communication; detecting anomalies, detecting typical profiles, comparing history data, data mining, big data algorithms, anticipating/forecasting maintenance tasks, production management, increasing energy efficiency.

The present disclosure allows measuring the hydrostatic pressure of a liquid contained, for example, in a barrel without having to open it, since the variation of the hydrostatic pressure between two points, one situated on the free surface of the liquid and another situated in the point from which the measurement is intended to be carried out (level measurement). Therefore, avoiding opening the barrel also translates into the following advantages: avoiding excessive gaseous exchanges leading to the excessive dissolution of oxygen leading to the formation of compounds, resulting from redox reactions, increasing the risk of loss of the quality potential or even the total loss of the product.

The present disclosure is related to the hydrostatic pressure, that is, it is related to the forces exerted by and on fluids at rest and relates to a level or density sensor device for barrels/sensorised stopper for barrels or vats, in particular a differential pressure sensor allowing for determining the level of a liquid contained in a vessel suitable for storing liquids, in particular, wine and also allowing for determining the density of said liquid contained in the vessel/barrel.

The hydrostatic pressure is determined by Stevin's theorem. The operating principle of the level measurement by the sensorised stopper is based on Stevin's theorem: P=ρgh wherein P corresponds to the hydrostatic pressure created by a column of liquid, ρ corresponds to the density of the liquid in kg/m³, g corresponds to the gravity acceleration in m/s², h corresponds to the liquid height/level above the measure point in meters. By measuring the value of P and considering that the value of ρ and g are constant after the calibration has been made, it is possible to obtain the value of h, which in this case represents the level of the liquid inside the barrel.

In order to obtain the value of the differential hydrostatic pressure in the liquid (ΔP) a differential pressure sensor is used, which measures the air or liquid pressure (FIG. 1).

Considering a point in the top of the barrel (in the air pocket) and another in the end of a tube dived into the liquid, the hydrostatic pressure is transmitted to the sensor through air that stays “captured” inside the tight tubes connected to the differential pressure sensor. Therefore, the differential pressure sensor compares the pressure between the air pressure inside the barrel and the air pressure in the tube end, with possibility of the differential pressure sensor being situated at the top of the barrel, namely integrated in a stopper, which simplifies the device construction and operation by sparing two sensors.

The equation necessary for measuring the density, in the case of using two tubes with a Δh is said Stevin's theorem. If the values of g and Δh are deemed constant and the value of ΔP is measured, it is possible to obtain the value of ρ, which in this case is the value of the liquid density.

The compensation of temperature causing the pressure of the air pocket to vary applies to both sides of the difference.

In an embodiment, if there is temperature variation inside the barrel, the air “captured” inside the measurement tubes expands, as a result of the temperature increase, or in opposition it contracts in case the temperature decreases. This leads to a variation of the hydrostatic pressure without a variation of the level, giving rise to a reading error.

This fact makes it necessary to acquire the temperature and compensate for it making the level measurement immune to that variation of the pressure inside the measurement tube.

In an embodiment, the “meniscus” effect is described and avoided, namely the weight thereof in the exact determination of the density, as well as other effects (dissolved gas). If the liquid inside the vessel releases gas (phenomenon occurring during the fermentation, resulting from the activity of yeasts), it may accumulate in the ends of the tube or tubes in case 2 tubes are dived in. The “meniscus” effect leads to the appearance of a “bubble” in the lower end of the measurement tube which releases itself whenever it has enough size for that to happen. This situation translates into a variation of the hydrostatic pressure without a variation of the level of the liquid or density thereof, thereby introducing a reading error. The bigger the “bubble” that can be accumulated by the “meniscus” effect, the bigger the introduced error. For minimizing this error, the end of the tubes must have such a shape that it minimizes the “meniscus” effect.

The present disclosure relates to a sensor device that may have the simultaneous function of vessel stopper or other format convenient to the installation, for measuring through a hydrostatic pressure difference, the volume or the density of a liquid contained in the vessel, wherein said sensor device comprises:
a main body;
a tube coupled to the main body for diving into the liquid regardless of whether the vessel is covered with the sensor device;
a hydrostatic pressure differential sensor placed in the main body;
wherein the hydrostatic pressure differential sensor has two sensor inlets, a first inlet tight connected to the upper top of said tube so that the tube maintains air present in the interior thereof when dived into the liquid, and a second inlet for communicating with the interior of the vessel.

The present disclosure also relates to a sensor device of hydrostatic pressure difference for measuring volume or density of a liquid contained in a vessel, wherein said sensor device comprises:
a main body;
a tube for diving into the liquid;
a hydrostatic pressure differential sensor;
an air injector for injecting air into said tube;
wherein the tube is coupled to the main body;
wherein the hydrostatic pressure differential sensor has two inlets, a first airtight inlet connected to the upper top of said tube, configured so that the tube maintains air present in the interior thereof when dived into the liquid, and a second inlet for communicating with the interior of the vessel.

In an embodiment, it is foreseen the introduction of air into the measurement tubes (tube(s) for diving into the liquid), being that such air may be introduced through one or more air injectors, such as air pumps, bottles with compressed air or any other pressurization form.

The air injector allows for the air column and meniscus thereof to be reconstructed in a given measurement tube, before carrying out measure, for expelling the liquid that has introduced in the interior of the tube throughout time (for example, due to air leakages in the tube or in the pressure sensor); it also allows for expelling air from the tubes whenever necessary for removing any dirt, that has introduced in the tubes, through air injection.

In an embodiment, the sensor device may measure, through a hydrostatic pressure difference, the density of the liquid contained in the vessel wherein said sensor device may comprise:
a second tube coupled to the main body for diving into the liquid, being that the length of the second tube is different from the length of the first tube and wherein the second inlet of the hydrostatic pressure differential sensor is airtight-connected to the upper top of the second tube so that the second tube maintains air present in the interior thereof when dived into the liquid.

In an embodiment, the sensor device may measure the volume and density of the liquid contained in the vessel, being that the sensor device may comprise:
a second tube coupled to the main body for diving into the liquid, being that the length of the second tube is different from the length of the first tube and a second hydrostatic pressure differential sensor;
wherein the second hydrostatic pressure differential sensor may have two inlets,
a first inlet of the second differential sensor airtight-connected to the upper top of the first tube, configured so that the tube maintains air present in the interior thereof when dived into the liquid, and a second inlet of the second differential sensor tight connected to the upper top of the second tube so that the second tube maintains air present in the interior thereof when dived into the liquid.

In an embodiment, the first or the second differential hydrostatic pressure differential sensor may comprise two non-differential pressure sensors and a differentiator for providing the difference between the pressures measured by the two non-differential sensors.

In an embodiment, the sensor device disclosed herein may comprise an additional tube for protecting the second inlet of the first hydrostatic pressure differential sensor.

In an embodiment, one or more of each tube, of all the tubes, has an air injector for injecting air into the corresponding tube.

In an embodiment, the sensor device herein disclosed may comprise an electronic data processor connected to the hydrostatic pressure differential sensor and to the air injector, wherein said processor is configured for injecting air into said tube before obtaining a measure by the hydrostatic pressure differential sensor, in particular said processor is configured for injecting air into said tube immediately before obtaining a measure by the hydrostatic pressure differential sensor.

In an embodiment, the tube or tubes may have an inferior end angle-cut for avoiding or minimizing meniscus effect of the liquid in the inferior end of the tube or tubes, the inferior end may be angle-cut at 30-60°, preferably 45°.

In an embodiment, the tube or tubes may have an inferior end angle-cut for avoiding or minimizing meniscus effect of the liquid in the inferior end of the tube or tubes.

In an embodiment, one or more tubes may have a superior end angle-coupled to the main body for avoiding or minimizing the meniscus effect of the liquid in the inferior end of the tube or tubes.

In an embodiment, the tube or tubes may be stainless, Teflon or plastic tube or tubes.

In an embodiment, the sensor device may comprise a temperature sensor coupled to the main body or to a said tube.

In an embodiment, the sensor device may comprise a sensor for measuring pH, turbidity, color, sugar amounts or alcohol concentration of the liquid, or combinations thereof, coupled to the main body or to a said tube.

In an embodiment, the sensor device may be cylindrical, quadrangular section prismatic, rectangular section prismatic, or frusto-conical.

In an embodiment, the sensor device may comprise a wireless transmitter for transmitting data collected by the sensor or sensors of pH, turbidity, color, sugar amounts or alcohol concentration of the liquid, or combinations thereof, coupled to the main body or to a said tube, being that the collected data may be transmitted periodically.

In an embodiment, the liquid may be wine, sparkling wine, juice, or a spirit drink.

In an embodiment, the vessel may be a barrel, tun, cask or vat.

In an embodiment, the sensor device herein disclosed may be a stopper of said vessel, for example wherein the vessel may be a barrel, tun, cask or vat.

The present disclosure also relates to a barrel, a tun, a cask or vat that may comprise the sensor device disclosed herein.

The present disclosure further relates to a system for measuring volume and density of a liquid contained in vessels comprising a plurality of sensor devices herein disclosed.

In an embodiment, said system may comprise an electronic data processor that may be configured for receiving, processing and storing the data collected by the sensor device.

In an embodiment, said processor may generate and send an alert for at least one user via email or SMS.

Throughout the description and claims the word “comprises” and variations of the word “comprises” are not intended to exclude other technical features, as other components or steps. Additional objects, advantages and features of the disclosure will become clear to those skilled in the art upon examination of the description or may be learned by practice of the invention. The following examples and figures are for illustrating the description and should not be limiting the scope of the disclosure. Furthermore, the present disclosure covers all possible combinations of specific or referential embodiments herein described.

### BRIEF DESCRIPTION OF THE DRAWINGS

Figures are attached for an easier understanding of the present disclosure that represents preferred embodiments for illustrating the description and should not be limiting the scope of the disclosure.

FIG. 1: Schematic representation of the operation of the differential sensor device used.

FIG. 2: Side view schematic representation of the assembly for measuring the level.

FIG. 3: Side view schematic representation of the set for measuring the density.

FIG. 4: Side view schematic representation of the set for measuring the level and density.

FIG. 5: Side view schematic representation of the set for measuring the level placed in the barrel.

FIG. 6: Side view schematic representation of the set for measuring the density placed in the barrel.

FIG. 7: Side view schematic representation of the set for measuring the level and density placed in the barrel.

FIG. 8: Bottom view schematic representation of the set for measuring the level or density.

FIG. 9: Bottom view schematic representation of the set for measuring the level and density.

FIG. 10: Schematic representation of the system with pumps for introducing air or air injectors.

FIG. 11: Side view schematic representation of the set for measuring the density placed in the vat.

FIG. 12: Schematic representation of the embodiment for preventing the “meniscus” effect by the angle-cut of the inferior end of the measurement tube.

FIG. 13: Schematic representation of the integrated system herein disclosed.

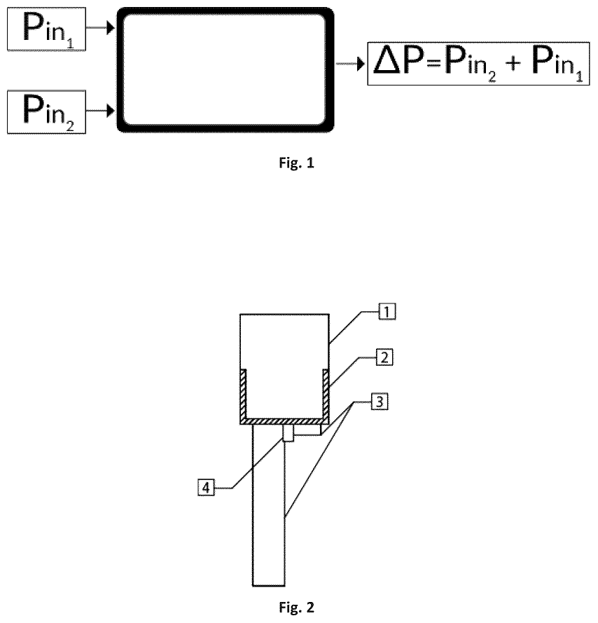

### DETAILED DESCRIPTION

FIG. 2 represents an embodiment for measuring the level of the liquid contained in the barrel, disclosing some of the components comprised in the present disclosure. FIG. 2 represents in particular a side view of the present disclosure wherein 1 represents the carcass/main body of the stopper/sensor device, 2 represents a silicone joint that may or may not exist and may or may not play the additional role of acting as a stopper (tightness of the vessel; 3 represents two tubes connected to the sensor, one for measuring the air pocket pressure and another with air in the interior thereof for measuring the liquid pressure at a given depth, in particular tubes that may comprise in the composition thereof a material compatible with the liquid with which stainless steel, Teflon or plastic is in contact; and 4 represents a temperature sensor.

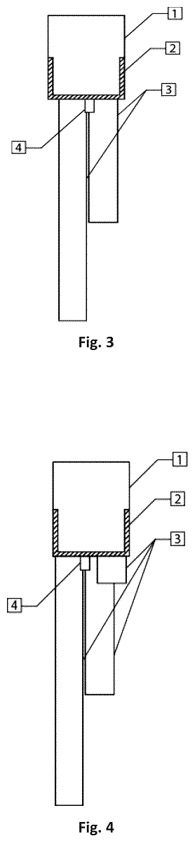

FIG. 3 represents an embodiment, it represents a side view of the set disclosed herein for measuring the liquid density contained in the barrel, with two tubes connected to the sensor with air in the interior thereof for measuring the liquid pressure at two predetermined depths.

FIG. 4 represents an embodiment of the present disclosure, it represents a side view of the set for measuring the liquid level and density contained in the barrel, with three tubes connected to the sensor, one for measuring the air pocket pressure and two tubes with air in the interior thereof for measuring the liquid pressure at two predetermined depths.

The tube for measuring the pressure in the air pocket in the described embodiments is optional, since the sensor will function equally well without it. The tube has here a physical protection function of the sensor.

The temperature sensor is optional, in case the temperature measure is not used or is provided by another sensor, inside or outside the barrel.

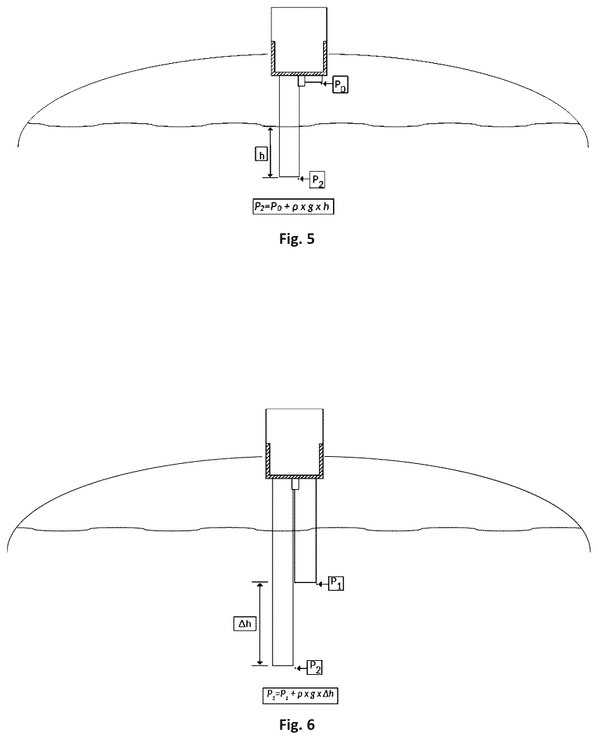

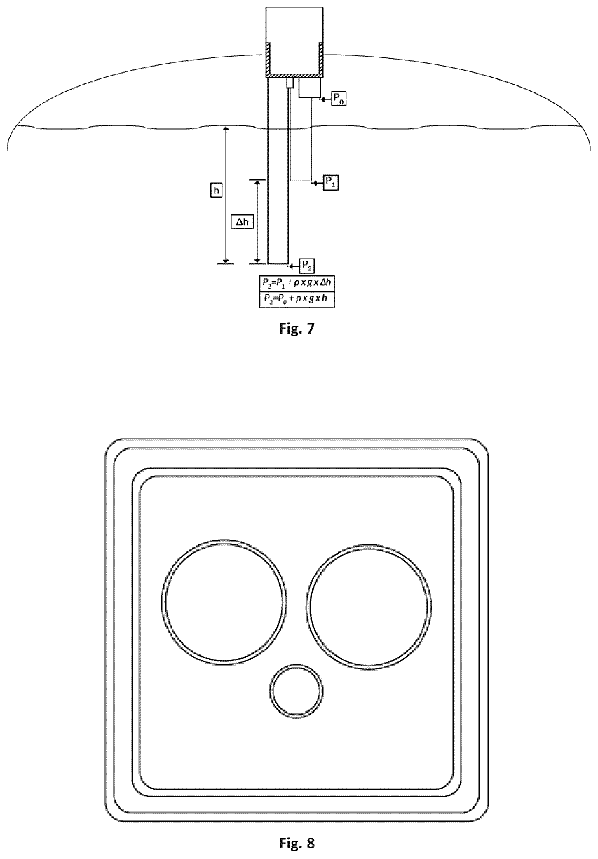

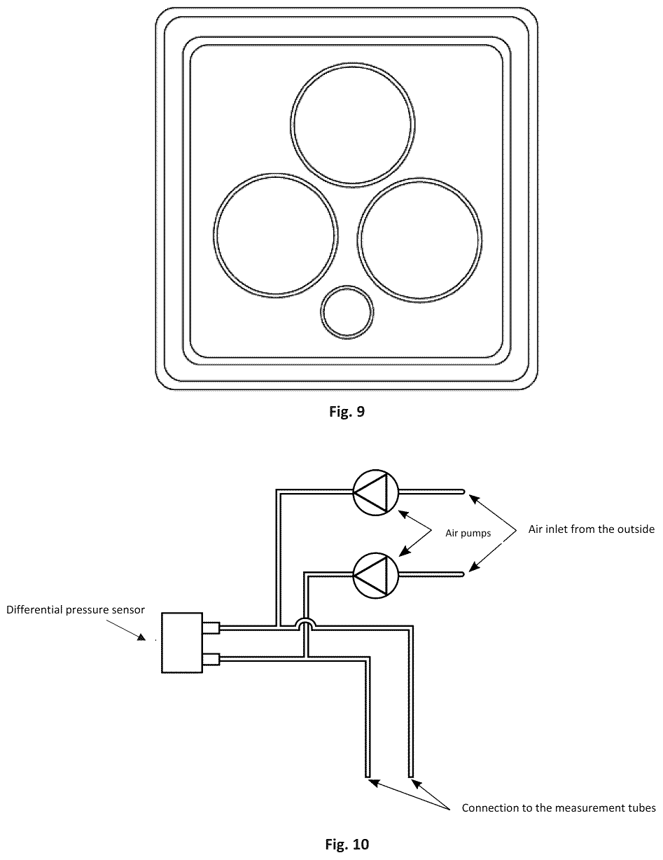

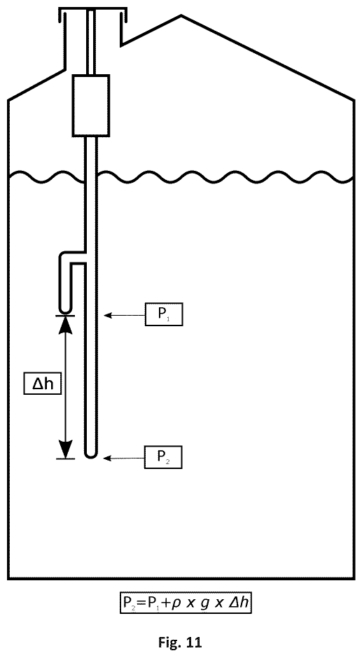

FIG. 5 represents an architecture of the monitoring platform, in particular a side view of the measurement set in the level placed in the barrel, being that P₀ represents the hydrostatic pressure measured in the interior of the vessel; P₂ represents the hydrostatic pressure measured in the tube end (first tube) that is dived into the liquid contained in the barrel and h represents the height between the point where the measurement of P₂ is made and the surface of the liquid.

FIG. 6 represents the architecture of the monitoring platform in particular a side view of the set for density measurement placed in the barrel wherein P₂ represents the hydrostatic pressure measured in the tube end (first tube) that is dived into the liquid contained in the barrel, P₁ represents the hydrostatic pressure measured in the end of a second tube that is dived into the liquid contained in the barrel and Δh represents the height difference between the points P₁ and P₂. FIG. 6 represents a joint embodiment of the level and density sensor.

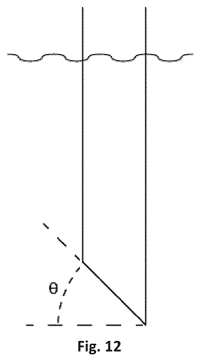

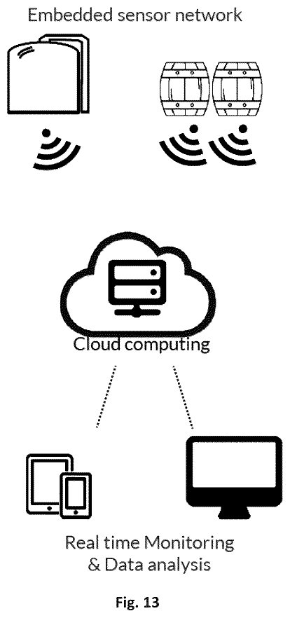

FIG. 12 represents an embodiment allowing for reducing or minimizing the meniscus effect that may introduce reading errors, either of liquid level or density level. Therefore for avoiding or minimizing the meniscus effect, each tube may comprise in the inferior end thereof an angle-cut, for example between 30-60°, preferably 45°, being that this angle is measured between the inferior opening plan of the tube and the liquid surface. Alternatively, or complementarily, the tube may be tilted in relation to the vertical.

In an embodiment, the initial calibration may be carried out by the following steps:
carrying out a measure with the sensor outside the liquid, that is in the air;
carrying out a measure with the sensor dived into the liquid d/2 mm (where d represents the total length of the sensor in mm, that is, it represents the maximum value of the level the sensor can measure);
carrying out a measure with the sensor dived into the liquid d mm (where d represents the maximum value of the level the sensor can measure).

In an embodiment, the initial calibration may also be carried out by the following steps:
carrying out a measure with the sensor outside the liquid, that is in the air;
carrying out a measure with the sensor dived into the liquid d/3 mm (where d represents the maximum value of the level the sensor can measure);
carrying out a measure with the sensor dived into the liquid 2×d/3 mm (where d represents the maximum value of the level the sensor can measure);
carrying out a measure with the sensor dived into the liquid d mm (where d represents the maximum value of the level the sensor can measure).

In an embodiment, the initial calibration carried out in 4 steps is a more precise calibration than the calibration carried out in 3 steps because more points are obtained giving rise to a better adjustment equation, decreasing the error associated to the calibration.

Although merely particular embodiments of the present disclosure have been represented and described herein, those skilled in the art will know how to introduce modifications and replace some technical features with equivalent ones, depending on the requisites of each situation, without departing from the scope of protection defined by the appended claims. The term “comprises” or “comprising” when used in this document is intended to indicate the presence of the mentioned features, elements, integers, steps and components, but not to preclude the presence or addition of one or more other features, integers, steps and components or combinations thereof. The following claims additionally set out embodiments of the disclosure.

What is claimed is:

1-22. (canceled)

23. A system comprising:
a first stainless steel tube that is straight between a first end and a second end; and
a first sensor, operably coupled to the first end of the first stainless steel tube, configured to measure a volume of liquid in which the second end of the first stainless steel tube is submerged, wherein:
the first stainless steel tube is tilted in relation to vertical to form an angle as measured between an opening at the second end of the first stainless steel tube and a surface of the liquid, and
the angle is greater than 30° and less than 60°.

24. The system of claim 23, wherein:
the first sensor is a hydrostatic pressure differential sensor.

25. The system of claim 24, wherein:
a first inlet of the hydrostatic pressure differential sensor is operably coupled to the first end of the first stainless steel tube,
a first air injector is configured to maintain air in the first stainless steel tube while the second end of the first stainless steel tube is in contact with the liquid, and
the volume of liquid is measured according to pressures at the first inlet and a second inlet.

26. The system of claim 25, wherein:
a second inlet of the hydrostatic pressure differential sensor is in contact with an interior of a vessel holding the liquid, and
the second inlet of the hydrostatic pressure differential sensor is in contact with air.

27. The system of claim 25, wherein the system comprises:
a second stainless steel tube for protecting the second inlet of the hydrostatic pressure differential sensor.

28. The system of claim 27, wherein:
a length of the second stainless steel tube is different from a length of the first stainless steel tube,
the second inlet of the hydrostatic pressure differential sensor is in contact with a first end of the second stainless steel tube, and
the second end of the second stainless steel tube is submerged into the liquid.

29. The system of claim 28, wherein the system comprises:
a second air injector configured to inject air into the first end of the second stainless steel tube.

30. The system of claim 24, wherein:
the hydrostatic pressure differential sensor comprises two non-differential pressure sensors and a differentiator for providing the difference between the pressures measured by the two non-differential sensors.

31. The system of claim 24, wherein:
the hydrostatic pressure differential sensor comprises an electronic data processor coupled to an air injector, and
the electronic data processor is configured to inject air into the first stainless steel tube before obtaining a measure by the hydrostatic pressure differential sensor.

32. The system of claim 23, wherein:
the second end of the first stainless steel tube is cut at angle that is greater than 30° and less than 60°.

33. The system of claim 23, wherein the system comprises:
a temperature sensor coupled to the first stainless steel tube, and
the measurement of the volume of the liquid varies according to a temperature measured by the temperature sensor.

34. The system of claim 23, wherein the system comprises:
one or more sensors for measuring pH, turbidity, color, sugar and/or alcohol concentration of the liquid.

35. The system of claim 23, wherein:
the sensor is configured to measure a density of the liquid.

36. The system of claim 23, wherein the system comprises:
a wireless transmitter for transmitting data collected by the sensor.

37. The system of claim 36, wherein the collected data are transmitted periodically.

38. The system of claim 23, wherein the system comprises:
a processor configured to store data collected by the sensor.

39. The system of claim 23, wherein the system comprises:
a sound alarm that is operable according to data collected by the sensor.

40. The system of claim 23, wherein the system comprises:
a visual alarm that is operable according to data collected by the sensor.

41. The system of claim 23, wherein:
the system is wirelessly coupled to a remote alarm that is operable according to data collected by the sensor.

42. A method comprising:
inserting a tube into liquid; and
measuring, via a sensor, a volume of the liquid, wherein:
the tube is straight between a first end and a second end,
the sensor is operably coupled to the first end of the tube,
the second end of the tube is submerged in the liquid,
the tube is tilted in relation to vertical to form an angle as measured between an opening at the second end of the tube and a surface of the liquid, and
the angle is greater than 30° and less than 60°.
